(19) United States
(12) Patent Application Publication
(10) Pub. No.: US 2017/0319212 A1
(43) Pub. Date: Nov. 9, 2017

(54) ELASTIC DEVICE FOR RECONSTRUCTING ROTATOR CUFFS

(71) Applicant: Antonio SAMBUSSETI, Cremona (cr) (IT)

(72) Inventor: Antonio SAMBUSSETI, Cremona (cr) (IT)

(21) Appl. No.: 15/526,203

(22) PCT Filed: Nov. 12, 2015
(86) PCT No.: PCT/IB2015/058748

§ 371 Date: May 11, 2017

(30) Foreign Application Priority Data
Nov. 13, 2014 (IT) MI2014A001960

(51) Int. Cl. A61B 17/11; A61F 2/00; A61L 27/50; A61L 27/18; A61L 27/08

(52) U.S. Cl. CPC A61B 17/1146; A61L 27/18; A61L 27/08; A61L 27/50; A61F 2/0063; A61F 2210/0076; A61L 2430/10

(57) ABSTRACT

A device for the reconstruction of the rotator cuff including a flat element having at least two opposite ends and adapted to be connected to at least one of the damaged tendons of the rotator cuff of a patient at the ends; the flat element is made of a multilayer silicone membrane.

The object of the present invention is a device for the reconstruction of the rotator cuff.

The present invention has application in the cases of damage to one or more points of the muscle-tendon complex of the shoulder articulation, which commonly takes the name “rotator cuff.”

As is known, devices exist which are fixed by means of suture to the damaged muscle-tendon tissue of the shoulder articulation (rotator cuff), in order to restore the functionality of the articulation.

Typically, these devices are made of non-resorbable and rigid materials. By way of example, the known devices are made of polyethylene terephthalate fibers, i.e. Dacron®. In this case, these temporarily restore the functionality of the damaged muscle-tendon tissue during the lesion healing phase. Subsequently, they remain in place and are not removed.

Disadvantageously, the materials used for making the known devices have a rigidity such to create a certain number of problems.

First of all, the excessive rigidity of the known device can cause undesired separations of the device during the convalescence phase. Clearly, in such unfavorable situation, it is necessary to once again subject the patient to a further surgical operation in order to restore the connection of the device and allow the healing of the damaged tissue.

In addition, the excessive rigidity of the device can cause limitations in the movement of the articulation of the patient even after the completion of the healing phase.

In this context, the technical task underlying the present invention is to propose a device for the reconstruction of the rotator cuff that overcomes the abovementioned drawback of the prior art.

In particular, the object of the present invention is to provide a device for the reconstruction of the rotator cuff that ensures a reliable connection to the muscle-tendon tissue and prevents undesired separations during convalescence.

The specified technical task and the specified object are substantially reached by a device for the reconstruction of the rotator cuff comprising the technical characteristics set forth in one or more of the enclosed claims.

Further characteristics and advantages of the present invention will be clearer from the exemplifying and therefore non-limiting description of a preferred but not exclusive embodiment of a device for the reconstruction of the rotator cuff, as illustrated in the enclosed drawings in which:

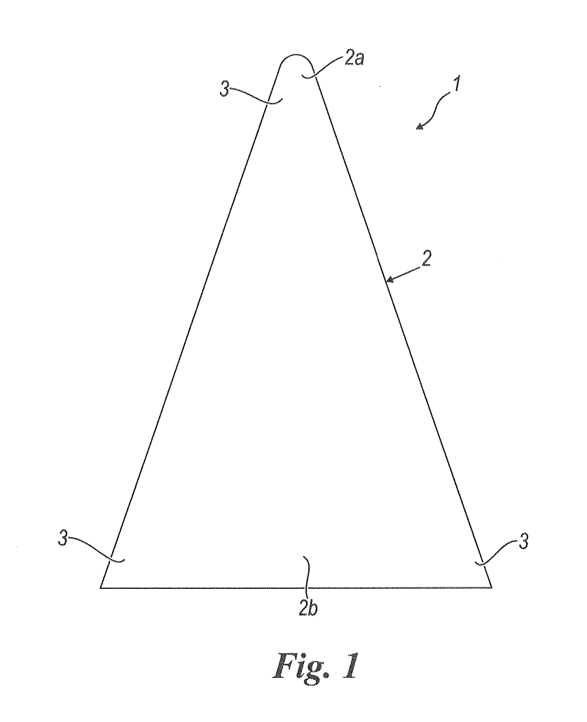

FIG. 1 is a schematic plan view of a device for the reconstruction of the rotator cuff.

With reference to the enclosed drawing, reference number 1 overall indicates a device for the reconstruction of the rotator cuff in accordance with the present invention.

The device 1 comprises a flat element 2, which has at least one first 2a and one second 2b end that are opposite each other.

The flat element 2, during use, is fixed by means of resorbable suture to the damaged muscle-tendon tissue of the rotator cuff.

The flat element 2 has a substantially wedge-shaped form.

In other words, the flat element 2 has a substantially triangular form in plan view. In particular, the flat element 2 has the form of an isosceles triangle.

The vertex at the equivalent sides is rounded.

The vertex at the equivalent sides defines the first end 2a of the flat element 2.

The portion of flat element 2 at the base side opposite the vertex instead defines the second end 2b.

According to the present invention, the flat element 2 is made by means of a multilayer silicone membrane.

The membrane of the flat element 2 has a thickness comprised between 500 μm and 700 μm, preferably the thickness of the membrane is substantially 600 μm.

In a preferred embodiment, the membrane comprises substantially 20 layers, each with thickness of about 30 μm.

In such a manner, the flat element 2 is therefore sufficiently flexible to be adapted to the movements of the articulation without risking separations of the device 1 itself from the tissues on which it is fixed.

The membrane is produced by means of a process illustrated in the patent application WO 2007/039159, which is incorporated herein for reference purposes.

By way of example, the silicone used can be constituted by dimethyl- and methyl vinyl-siloxane copolymers, with silicon reinforcements.

Advantageously, the silicone can have radio-opacifying additives such as barium sulfate, titanium dioxide or the like, in a manner such that the endoprosthesis 1 can be detected by means of radiological diagnostic techniques.

Respective layers of highly biocompatible protection material are applied on both surfaces of the flat elements 2. By way of example, such material is pyrolytic turbostratic carbon with a thickness comprised between 0.2 μm and 0.3 μm.

In both embodiments, the device 1 has fixing areas 3 placed at the ends 2a, 2b of the flat element 2 for fixing, through resorbable suture, the flat element 2 to the damaged muscle-tendon tissue.

In particular, the fixing areas 3 are arranged at the vertices of the flat element 2.

The invention attains the preset object.

Indeed, the device for the reconstruction of the rotator cuff has characteristics of flexibility and elasticity such to be easily adapted to articular movements, without any risk of separation and hence without the risk of having to once again operate in order to restore the device.

1. A device for the reconstruction of the rotator cuff comprising a flat element having at least two opposite ends and is adapted to be connected to at least one of the damaged tendons of the rotator cuff of a patient at said ends; wherein the flat element is made of a multilayer silicone membrane.

2. The device according to claim 1, wherein the multilayer silicone membrane of the flat element has a thickness between 500 μm and 700 μm.

3. The device according to claim 2, wherein the membrane of the flat element comprises a number of layers between 18 and 22; each layer having a thickness substantially of 30 μm.

4. The device according to claim 1, wherein the silicone of said membrane comprises dimethyl- and methyl vinyl-siloxane copolymers.

5. The device according to claim 4, wherein at least one face of the flat element also comprises a protective layer made of pyrolytic turbostratic carbon.

6. The device according to claim 5, wherein both faces of the flat element also comprise a protective layer made of pyrolytic turbostratic carbon.

7. The device according to claim 5, wherein the layer of pyrolytic turbostratic carbon has a thickness comprised between 0.2 μm and 0.3 μm.

8. The device according to claim 1, wherein it has fixing areas placed at the ends of the flat element adapted to be employed for fixing, through resorbable suture, said flat element to said tendon.

9. The device according to claim 1, wherein the membrane of the flat element has a thickness comprised about 600 μm.

10. The device according to claim 2, wherein the membrane of the flat element comprises a number of layers comprises 20 layers; each layer having a thickness substantially of 30 μm.

11. The device according to claim 1, wherein the silicone of said membrane comprises dimethyl- and methyl vinyl-siloxane copolymers with silicon reinforcements.

12. The device according to claim 2, wherein the membrane of the flat element comprises between 18 and 22 layers.

13. The device according to claim 6, wherein the layer of pyrolytic turbostratic carbon has a thickness comprised between 0.2 μm and 0.3 μm.

14. The device according to claim 13, wherein the membrane of the flat element comprises a number of layers between 18 and 22, wherein each said layer of pyrolytic turbostratic carbon has a thickness comprised between 0.2 μm and 0.3 μm.

15. The device according to claim 14, wherein said flat element has an isosceles triangle form in plan view.

16. The device according to claim 15, wherein the triangle form has equivalent sides and a vertex of the equivalent sides is rounded.
